(19) United States
(12) Patent Application Publication
(10) Pub. No.: US 2026/0241178 A1
(43) Pub. Date: Aug. 20, 2026

(54) Automated Creation of Stimulation Programs for Spinal Cord Stimulation

(71) Applicant: Boston Scientific Neuromodulation Corporation, Valencia, CA (US)

(72) Inventors: G. Karl Steinke, Valencia, CA (US); Kacey Auten, Bend, OR (US); Nina Pehler, Edina, MN (US); Nirmal Shah, Palmdale, CA (US); Ryan Schwoerer, Lino Lakes, MN (US); Dennis Vansickle, Lancaster, CA (US); Tianhe Zhang, Studio City, CA (US); Dheerendra Kashyap, Valencia, CA (US); Radha Kasireddy, Valencia, CA (US)

(21) Appl. No.: 19/446,508

(22) Filed: Jan. 12, 2026

Related U.S. Application Data
(60) Provisional application No. 63/758,576, filed on Feb. 14, 2025.

(51) Int. Cl. A61N 1/372; A61N 1/36

(52) U.S. Cl. CPC A61N 1/37247; A61N 1/36157; A61N 1/36185; A61N 1/37264; A61N 1/37282

(57) ABSTRACT

Systems for programming a neurostimulator for providing electrical neurostimulation to treat a patient are described. The systems are configured to allow a user, typically a clinician, to configure a source stimulation program. The system then uses the source program and/or other inputs to suggest additional stimulation programs (i.e., derivative stimulation programs) that may be created and used to program the patient’s neurostimulator. The other inputs may specify information about the patient, the patient's or clinician’s goals and priorities during the programming session, and/or information about the patient's neurostimulator, such as whether the neurostimulator uses a primary cell or rechargeable battery. The system can execute algorithms to use the source program and/or the other inputs to automatically create the derivative stimulation programs and to program the neurostimulator with those programs.

This is a non-provisional application of U.S. Provisional Patent Application Serial No. 63/758,576, filed February 14, 2025, which is incorporated herein by reference in its entirety, and to which priority is hereby claimed.

### FIELD OF THE INVENTION

This application relates to Implantable Medical Devices (IMDs), generally, Spinal Cord Stimulators, more specifically, and to methods of programming such devices.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Patent 6,516,227. However, the present invention may find applicability with any implantable neurostimulator device system, such as a deep brain stimulation (DBS) system, for example.

An SCS system typically includes a neurostimulator, such as an Implantable Pulse Generator (IPG) 10 shown in FIG. 1. The IPG 10 includes a biocompatible device case 12 that holds the circuitry and battery 14 necessary for the IPG to function. The IPG 10 is coupled to electrodes 16 via one or more electrode leads 15 that form an electrode array 17 In the context of SCS, the lead(s) carrying the electrode array are implanted within the patient’s spinal column such that the electrodes are maintained near the patient’s spinal cord. The electrodes 16 are configured to contact a patient’s tissue and are carried on a flexible body 18, which also houses the individual lead wires 20 coupled to each electrode 16. The lead wires 20 are also coupled to proximal contacts 22, which are insertable into lead connectors 24 fixed in a header 23 on the IPG 10, which header can comprise an epoxy for example. Once inserted, the proximal contacts 22 connect to header contacts within the lead connectors 24, which are in turn coupled by feedthrough pins through a case feedthrough to circuitry within the case 12, although these details aren’t shown.

In the illustrated IPG 10, there are sixteen lead electrodes (E1-E16) split between two leads 15, with the header 23 containing a 2x1 array of lead connectors 24. However, the number of leads and electrodes in an IPG is application specific and therefore can vary. The conductive case 12 can also comprise an electrode (Ec). In a SCS application, the electrode leads 15 are typically implanted proximate to the dura in a patient’s spinal column on the right and left sides of the spinal cord midline. The proximal electrodes 22 are tunneled through the patient’s tissue to a distant location such as the buttocks where the IPG case 12 is implanted, at which point they are coupled to the lead connectors 24. In other IPG examples designed for implantation directly at a site requiring stimulation, the IPG can be lead-less, having electrodes 16 instead appearing on the body of the IPG for contacting the patient’s tissue. The IPG leads 15 can be integrated with and permanently connected the case 12 in other IPG solutions. The goal of SCS therapy is to provide electrical stimulation from the electrodes 16 to alleviate a patient’s symptoms, most notably chronic back pain.

IPG 10 can include an antenna 26a allowing it to communicate bi-directionally with a number of external devices, as shown in FIG. 4. The antenna 26a as depicted in FIG. 1 is shown as a conductive coil within the case 12, although the coil antenna 26a can also appear in the header 23. When antenna 26a is configured as a coil, communication with external devices preferably occurs using near-field magnetic induction. IPG may also include a Radio-Frequency (RF) antenna 26b. In FIG. 1, RF antenna 26b is shown within the header 23, but it may also be within the case 12. RF antenna 26b may comprise a patch, slot, or wire, and may operate as a monopole or dipole. RF antenna 26b preferably communicates using far-field electromagnetic waves. RF antenna 26b may operate in accordance with any number of known RF communication standards, such as Bluetooth, Zigbee, WiFi, MICS, and the like.

Stimulation in IPG 10 is typically provided by pulses, as shown in FIG. 2. Stimulation parameters typically include the amplitude of the pulses (A; whether current or voltage); the frequency (F) and pulse width (PW) of the pulses; the electrodes 16 (E) activated to provide such stimulation; and the polarity (P) of such active electrodes, i.e., whether active electrodes are to act as anodes (that source current to the tissue) or cathodes (that sink current from the tissue). These stimulation parameters taken together comprise a stimulation program that the IPG 10 can execute to provide therapeutic stimulation to a patient.

In the example of FIG. 2, electrode E5 has been selected as an anode, and thus provides pulses which source a positive current of amplitude +A to the tissue. Electrode E4 has been selected as a cathode, and thus provides pulses which sink a corresponding negative current of amplitude -A from the tissue. This is an example of bipolar stimulation, in which only two lead-based electrodes are used to provide stimulation to the tissue (one anode, one cathode). However, more than one electrode may act as an anode at a given time, and more than one electrode may act as a cathode at a given time (e.g., tripole stimulation, quadripole stimulation, etc.).

The pulses as shown in FIG. 2 are biphasic, comprising a first phase 30a, followed quickly thereafter by a second phase 30b of opposite polarity. As is known, use of a biphasic pulse is useful in active charge recovery because the second phase 30b actively recovers the charge that is injected during the first phase 30a. For example, each electrodes’ current path to the tissue may include a serially-connected DC-blocking capacitor, see, e.g., U.S. Patent Application Publication 2016/0144183, which will charge during the first phase 30a and discharged (be recovered) during the second phase 30b. In the example shown, the first and second phases 30a and 30b have the same duration and amplitude (although opposite polarities), which ensures the same amount of charge during both phases. However, the second phase 30b may also be charged balance with the first phase 30a if the integral of the amplitude and durations of the two phases are equal in magnitude, as is well known. The width of each pulse, PW, is defined here as the duration of first pulse phase 30a, although pulse width could also refer to the total duration of the first and second pulse phases 30a and 30b as well. Note that an interphase period (IP) during which no stimulation is provided may be provided between the two phases 30a and 30b. Waveforms such as those shown in FIG. 2 may be referred to as “active recharge” waveforms. It should be noted that stimulation may also be applied using passive recharge waveforms wherein the charge injected during the first phase (e.g. 30a) is not actively recovered during an active second phase. Instead, the injected charge is recovered by connecting the electrode node(s) to a common voltage through a resistance, thereby allowing the injected charge to passively drain to the common voltage. Passive charge recovery (i.e., passive recharge) is well known and is not explained here in detail. The reader is referred to U.S. Patent No. 10,716,937 (“the ‘937 Patent”), the entire contents of which are incorporated herein by reference, for an explanation of a system for providing variable and programmable resistance during passive charge recovery.

IPG 10 includes stimulation circuitry 28 that can be programmed to produce the stimulation pulses at the electrodes as defined by the stimulation program. Stimulation circuitry 28 can for example comprise the circuitry described in U.S. Patent Application Publications 2018/0071513 and 2018/0071520, or in USPs 8,606,362 and 8,620,436. These references are incorporated herein by reference.

FIG. 3 shows an external trial stimulation environment that may precede implantation of an IPG 10 in a patient. During external trial stimulation, stimulation can be tried on a prospective implant patient without going so far as to implant the IPG 10. Instead, one or more trial leads 15’ are implanted in the patient’s tissue 32 at a target location 34, such as within the spinal column as explained earlier. The proximal ends of the trial lead(s) 15’ exit an incision 36 and are connected to an External Trial Stimulator (ETS) 40. The ETS 40 generally mimics operation of the IPG 10, and thus can provide stimulation pulses to the patient’s tissue as explained above. See, e.g., 9,259,574, disclosing a design for an ETS. The ETS 40 is generally worn externally by the patient for a short while (e.g., two weeks), which allows the patient and his clinician to experiment with different stimulation parameters to try and find a stimulation program that alleviates the patient’s symptoms (e.g., pain). If external trial stimulation proves successful, trial lead(s) 15’ are explanted, and a full IPG 10 and lead(s) 15 are implanted as described above; if unsuccessful, the trial lead(s) 15’ are simply explanted.

Like the IPG 10, the ETS 40 can include one or more antennas to enable bi-directional communications with external devices, explained further with respect to FIG. 4. Such antennas can include a near-field magnetic-induction coil antenna 42a, and/or a far-field RF antenna 42b, as described earlier. ETS 40 may also include stimulation circuitry 44 able to form the stimulation pulses in accordance with a stimulation program, which circuitry may be similar to or comprise the same stimulation circuitry 28 present in the IPG 10. ETS 40 may also include a battery (not shown) for operational power.

### SUMMARY

Disclosed herein is a system for programming a neurostimulator for providing neurostimulation for a patient, wherein the neurostimulator is configured to connect to one or more electrode leads implantable in the patient, the one or more electrode leads each comprising a plurality of electrodes, the system comprising: an external programming device comprising control circuitry configured to: receive a source stimulation program, receive an indication of one or more derivative stimulation programs which are different from the source stimulation program, for each of the one or more derivative stimulation programs, automatically cause the external device to make a template stimulation program, for each template stimulation program, automatically execute steps to modify the template stimulation program to yield one of the indicated derivative stimulation programs, and cause the external programming device to transmit at least the one or more derivative stimulation programs to an external controller for the neurotransmitter. According to some embodiments, each of the template stimulation programs comprise a copy of the source stimulation program. According to some embodiments, the neurostimulator is an implantable pulse generator (IPG) and the one or more electrode leads are configurable for implantation within the patient’s spinal column. According to some embodiments, the source stimulation program and the one or more derivative stimulation programs are each configured to provide pain relief for the patient. According to some embodiments, one or more parameters of one or more of the derivative programs is determined based on parameters of the source stimulation program. According to some embodiments, the control circuitry is configured to cause the external programming device to present a graphical user interface (GUI) comprising control elements configured to allow a user to create and modify the source stimulation program. According to some embodiments, the GUI comprises a menu of available derivative stimulation programs. According to some embodiments, the control circuitry is configured to generate the indication of the one or more derivative stimulation programs in response to user selections from the menu. According to some embodiments, the indication of one or more derivative stimulation programs is responsive to user selections from the menu. According to some embodiments, the available derivative stimulation programs are determined, at least in part, in response to one or more inputs. According to some embodiments, the one or more inputs are provided via the GUI. According to some embodiments, the one or more inputs are not provided via the GUI. According to some embodiments, the control circuitry is configured to associate metadata with each of the one or more inputs and to use the metadata to select the available derivative stimulation programs. According to some embodiments, the one or more inputs comprises information about the neurostimulator. According to some embodiments, the one or more inputs indicates if the power supply of the neurostimulator is rechargeable or is a primary cell. According to some embodiments, the one or more inputs comprises information about the patient. According to some embodiments, the one or more inputs comprises information about one or more of the etiology of the patient’s condition. According to some embodiments, the GUI is configured to guide a user through a process of entering the one or more inputs. According to some embodiments, guiding the user comprises displaying one or more selectable inputs indicative of the one or more inputs. According to some embodiments, guiding the user comprises using a chat tool to present questions to the user and to receive answers to the questions. According to some embodiments, the source stimulation program comprises an electrode configuration specifying a fraction of stimulation current provided at each electrode and a total amplitude of the stimulation current. According to some embodiments, the stimulation program specifies a total amplitude corresponding to the patient’s perception threshold. According to some embodiments, the source stimulation program is a sub-perception (sub-P) stimulation program. According to some embodiments, at least one of the one or more derivative stimulation programs is a supra-perception (supra-P) stimulation program. According to some embodiments, modifying the copy of the source stimulation program comprises executing a script that automatically emulates one or more steps that a user would perform using the GUI to modify the copy of the source stimulation program to create the respective derivative stimulation program. According to some embodiments, the one or more steps comprise changing an electrode configuration, wherein the electrode configuration specifies a fraction of stimulation current provided at each electrode. According to some embodiments, the one or more steps comprise moving a center point of stimulation (CPS). According to some embodiments, the one or more steps comprise changing a total amplitude of stimulation current. According to some embodiments, the source stimulation program is a bipolar stimulation program and wherein the one or more steps comprise changing the stimulation to monopolar stimulation. According to some embodiments, the control circuitry is configured to cause the GUI to present a calibration interface whereby the user can calibrate one or more of the derivative stimulation programs. According to some embodiments, the control circuitry is configured to calibrate one or more of an amplitude, stimulation field, pulse width, and/or rate of one or more of the derivate stimulation programs. According to some embodiments, the control circuitry is configured to cause the GUI to present a calibration interface whereby the user can compose a schedule comprising a first duration during which a first of the source or derivative stimulation programs is active to provide stimulation and a second duration during which a second of the source or derivative stimulation programs is active to provide stimulation.

Also disclosed herein is a non-transitory computer readable medium for configuring for configuring an external programming device for programming a neurostimulator for providing neurostimulation for a patient, wherein the neurostimulator is configured to connect to one or more electrode leads implantable in the patient, the one or more electrode leads each comprising a plurality of electrodes, wherein the non-transitory computer readable medium comprises instructions, which when execute by the external programming device configures the external programming device to: receive a source stimulation program, receive an indication of one or more derivative stimulation programs which are different from the source stimulation program, for each of the one or more derivative stimulation programs, automatically cause the external device to make a template stimulation program, for each template stimulation program, automatically execute steps to modify the template stimulation program to yield one of the indicated derivative stimulation programs, and cause the external programming device to transmit at least the one or more derivative stimulation programs to an external controller for the neurotransmitter. According to some embodiments, each of the template stimulation programs comprise a copy of the source stimulation program. According to some embodiments, the neurostimulator is an implantable pulse generator (IPG) and the one or more electrode leads are configurable for implantation within the patient’s spinal column. According to some embodiments, the source stimulation program and the one or more derivative stimulation programs are each configured to provide pain relief for the patient. According to some embodiments, the one or more parameters of one or more of the derivative programs is determined based on parameters of the source stimulation program. According to some embodiments, the external programming device is configured to cause the external programming device to present a graphical user interface (GUI) comprising control elements configured to allow a user to create and modify the source stimulation program. According to some embodiments, the GUI comprises a menu of available derivative stimulation programs. According to some embodiments, the external programming device is configured to generate the indication of the one or more derivative stimulation programs in response to user selections from the menu. According to some embodiments, the indication of one or more derivative stimulation programs is responsive to user selections from the menu. According to some embodiments, the available derivative stimulation programs are determined, at least in part, in response to one or more inputs. According to some embodiments, the one or more inputs are provided via the GUI. According to some embodiments, the one or more inputs are not provided via the GUI. According to some embodiments, the external programming device is configured to associate metadata with each of the one or more inputs and to use the metadata to select the available derivative stimulation programs. According to some embodiments, the one or more inputs comprises information about the neurostimulator. According to some embodiments, the one or more inputs indicates if the power supply of the neurostimulator is rechargeable or is a primary cell. According to some embodiments, the one or more inputs comprises information about the patient. According to some embodiments, the one or more inputs comprises information about one or more of the etiology of the patient’s condition. According to some embodiments, the GUI is configured to guide a user through a process of entering the one or more inputs. According to some embodiments, guiding the user comprises displaying one or more selectable inputs indicative of the one or more inputs. According to some embodiments, guiding the user comprises using a chat tool to present questions to the user and to receive answers to the questions. According to some embodiments, the source stimulation program comprises an electrode configuration specifying a fraction of stimulation current provided at each electrode and a total amplitude of the stimulation current. According to some embodiments, the stimulation program specifies a total amplitude corresponding to the patient’s perception threshold. According to some embodiments, the source stimulation program is a sub-perception (sub-P) stimulation program. According to some embodiments, at least one of the one or more derivative stimulation programs is a supra-perception (supra-P) stimulation program. According to some embodiments, modifying the copy of the source stimulation program comprises executing a script that automatically emulates one or more steps that a user would perform using the GUI to modify the copy of the source stimulation program to create the respective derivative stimulation program. According to some embodiments, the one or more steps comprise changing an electrode configuration, wherein the electrode configuration specifies a fraction of stimulation current provided at each electrode. According to some embodiments, the one or more steps comprise moving a center point of stimulation (CPS). According to some embodiments, the one or more steps comprise changing a total amplitude of stimulation current. According to some embodiments, the source stimulation program is a bipolar stimulation program and wherein the one or more steps comprise changing the stimulation to monopolar stimulation. According to some embodiments, the external programming device is configured to cause the GUI to present a calibration interface whereby the user can calibrate one or more of the derivative stimulation programs. According to some embodiments, the external programming device is configured to calibrate one or more of an amplitude, stimulation field, pulse width, and/or rate of one or more of the derivate stimulation programs. According to some embodiments, the external programming device is configured to cause the GUI to present a calibration interface whereby the user can compose a schedule comprising a first duration during which a first of the source or derivative stimulation programs is active to provide stimulation and a second duration during which a second of the source or derivative stimulation programs is active to provide stimulation.

Also disclosed herein is a system for programming a neurostimulator for providing neurostimulation to a patient, wherein the neurostimulator is configured to connect to one or more electrode leads implantable in the patient, the one or more electrode leads each comprising a plurality of electrodes, the system comprising: an external programming device comprising control circuitry configured to: present a graphical user interface (GUI) on a screen of the external programming device, receive, via the GUI, first user inputs defining parameters for a source stimulation program, create the source stimulation program using the first user inputs, receive second inputs, use the second inputs to select one or more available derivative stimulation programs from a library of available derivative stimulation programs, receive, via the GUI, a user selection of one or more of the available derivative stimulation programs, automatically create the selected one or more of the available derivative stimulation programs, and use the source stimulation program and the created derivative stimulation programs to program the neurostimulator. According to some embodiments, the second inputs comprise second user inputs received via the GUI. According to some embodiments, the second user inputs comprise information about the patient. According to some embodiments, the information about the patient comprises information about a condition of the patient. According to some embodiments, the information about the patient comprises information about a goal of the patient during the programming. According to some embodiments, the information about the patient comprises information about a medical condition of the patient. According to some embodiments, the GUI is configured to guide a user through a process of entering the second user inputs. According to some embodiments, the second inputs are not received via the GUI. According to some embodiments, the second inputs comprise information about the neurostimulator. According to some embodiments, the information about the neurostimulator comprises information about the neurostimulator’s power supply.

Features which are described in the context of separate aspects and embodiments of the invention may be used together and/or be interchangeable. Similarly, features described in the context of a single embodiment may also be provided separately or in any suitable sub-combination. The invention may also reside in the methods performed by the above-described systems, graphical user interfaces for facilitating those methods, and non-transitory computer readable media containing instructions for programming a system and/or an external device (via its control circuitry) for carrying out the methods, a programmed implantable pulse generator (IPG) or external trial stimulator (ETS), external pulse generator (EPG), (via their respective control circuitry) for carrying out the above methods, a system including a programmed external device and IPG or ETS for carrying out the above methods, or as a computer-readable media for carrying out the above methods stored in an external device or IPG or ETS.

### BRIEF DESCRIPTION OF THE DRAWINGS

FIG. 1 shows an Implantable Pulse Generator (IPG) useable for Spinal Cord Stimulation (SCS).

FIG. 2 shows an example of stimulation pulses producible by the IPG.

FIG. 3 shows use of an External Trial Stimulator (ETS) useable to provide stimulation before implantation of an IPG.

FIG. 4 shows various external devices capable of communicating with and programming stimulation in an IPG and ETS.

FIG. 5 shows a Graphical User Interface (GUI) of a clinician programmer external device for setting or adjusting stimulation parameters.

FIG. 6 shows sweet spot searching to determine effective electrodes for a patient using a movable sub-perception bipole.

FIG. 7 shows sweet spot searching to determine effective electrodes for a patient using a movable supra-perception bipole.

FIG. 8 shows an overview of creating derivative stimulation programs from a source stimulation program.

FIG. 9A shows an overview of a system for creating derivative stimulation programs from a source stimulation program; FIG. 9B shows an example of an algorithm for classifying a patient based on an expected outcome from a programming session.

FIG. 10 shows a GUI for deriving a source stimulation program.

FIG. 11 shows a GUI for selecting derivative stimulation programs.

FIG. 12 shows steps for creating derivative stimulation programs from a source stimulation program.

FIG. 13 shows a GUI for calibrating stimulation programs.

### DETAILED DESCRIPTION

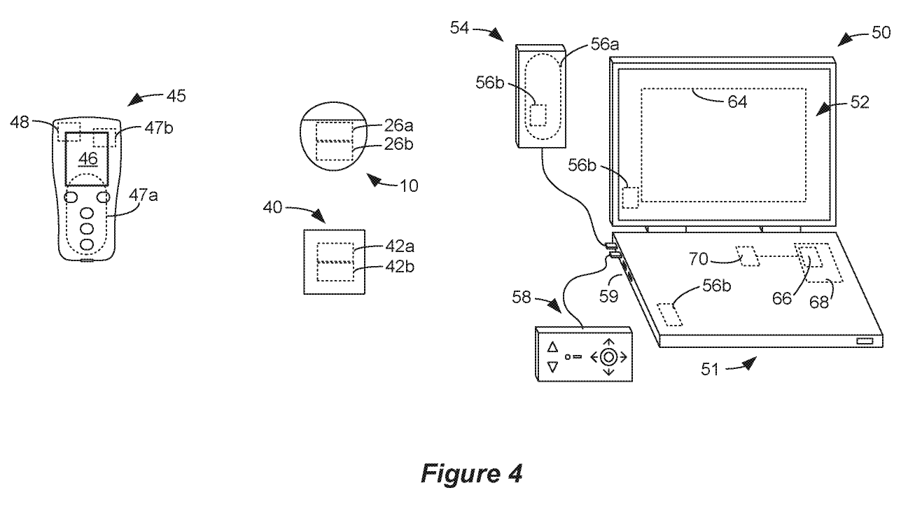

FIG. 4 shows various external devices that can wirelessly communicate data with the IPG 10 and the ETS 40, including a patient, hand-held external controller 45, and a clinician programmer (CP) 50. Both devices 45 and 50 can be used to send a stimulation program to the IPG 10 or ETS 40—that is, to program their stimulation circuitries 28 and 44 to produce pulses with a desired shape and timing described earlier. Both devices 45 and 50 may also be used to adjust one or more stimulation parameters of a stimulation program that the IPG 10 or ETS 40 is currently executing. Devices 45 and 50 may also receive information from the IPG 10 or ETS 40, such as various status information, etc.

External controller 45 can be as described in U.S. Patent Application Publication 2015/0080982 for example and may comprise a dedicated controller configured to work with the IPG 10. External controller 45 may alternatively comprise a general-purpose mobile electronics device such as a smartphone which has been programmed with a Medical Device Application (MDA) allowing it to work as a wireless controller for the IPG 10 or ETS 40, as described in U.S. Patent Application Publication 2015/0231402. External controller 45 includes a user interface, including means for entering commands (e.g., buttons or icons) and a display 46. The external controller 45’s user interface enables a patient to adjust stimulation parameters, although it may have limited functionality when compared to the more-powerful clinician programmer 50, described shortly.

The external controller 45 can have one or more antennas capable of communicating with the IPG 10 and ETS 40. For example, the external controller 45 can have a near-field magnetic-induction coil antenna 47a capable of wirelessly communicating with the coil antenna 26a or 42a in the IPG 10 or ETS 40. The external controller 45 can also have a far-field RF antenna 47b capable of wirelessly communicating with the RF antenna 26b or 42b in the IPG 10 or ETS 40.

The external controller 45 can also have control circuitry 48 such as a microprocessor, microcomputer, an FPGA, other digital logic structures, etc., which is capable of executing instructions an electronic device. Control circuitry 48 can for example receive patient adjustments to stimulation parameters and create a stimulation program to be wirelessly transmitted to the IPG 10 or ETS 40.

Clinician programmer 50 is described further in U.S. Patent Application Publication 2015/0360038 and is only briefly explained here. The clinician programmer 50 can comprise a computing device 51, such as a desktop, laptop, or notebook computer, a tablet, a mobile smart phone, a Personal Data Assistant (PDA)-type mobile computing device, etc. In FIG. 4, computing device 51 is shown as a laptop computer that includes typical computer user interface means such as a screen 52, a mouse, a keyboard, speakers, a stylus, a printer, etc., not all of which are shown for convenience. Also shown in FIG. 4 are accessory devices for the clinician programmer 50 that are usually specific to its operation as a stimulation controller, such as a communication “wand” 54, and a joystick 58, which are coupleable to suitable ports on the computing device 51, such as USB ports 59 for example.

The antenna used in the clinician programmer 50 to communicate with the IPG 10 or ETS 40 can depend on the type of antennas included in those devices. If the patient’s IPG 10 or ETS 40 includes a coil antenna 26a or 42a, wand 54 can likewise include a coil antenna 56a to establish near-filed magnetic-induction communications at small distances. In this instance, the wand 54 may be affixed in close proximity to the patient, such as by placing the wand 54 in a belt or holster wearable by the patient and proximate to the patient’s IPG 10 or ETS 40.

If the IPG 10 or ETS 40 includes an RF antenna 26b or 42b, the wand 54, the computing device 51, or both, can likewise include an RF antenna 56b to establish communication with the IPG 10 or ETS 40 at larger distances. (Wand 54 may not be necessary in this circumstance). The clinician programmer 50 can also establish communication with other devices and networks, such as the Internet, either wirelessly or via a wired link provided at an Ethernet or network port.

An important aspect of providing SCS therapy for a patient involves programming their stimulator device to provide the particular electrical stimulation that is expected to be therapeutically effective for treating the patient’s particular indication. This involves programming the particular electrodes that will deliver the stimulation and the particular stimulation patterns that will be delivered at those electrodes. To program stimulation programs or parameters for the IPG 10 or ETS 40, the clinician interfaces with a clinician programmer graphical user interface (GUI) 64 provided on the display 52 of the computing device 51. As one skilled in the art understands, the GUI 64 can be rendered by execution of clinician programmer software 66 on the computing device 51, which software may be stored in the device’s non-volatile memory 68. One skilled in the art will additionally recognize that execution of the clinician programmer software 66 in the computing device 51 can be facilitated by control circuitry 70 such as a microprocessor, microcomputer, an FPGA, other digital logic structures, etc., which is capable of executing programs in a computing device. Such control circuitry 70, in addition to executing the clinician programmer software 66 and rendering the GUI 64, can also enable communications via antennas 56a or 56b to communicate stimulation parameters chosen through the GUI 64 to the patient’s IPG 10.

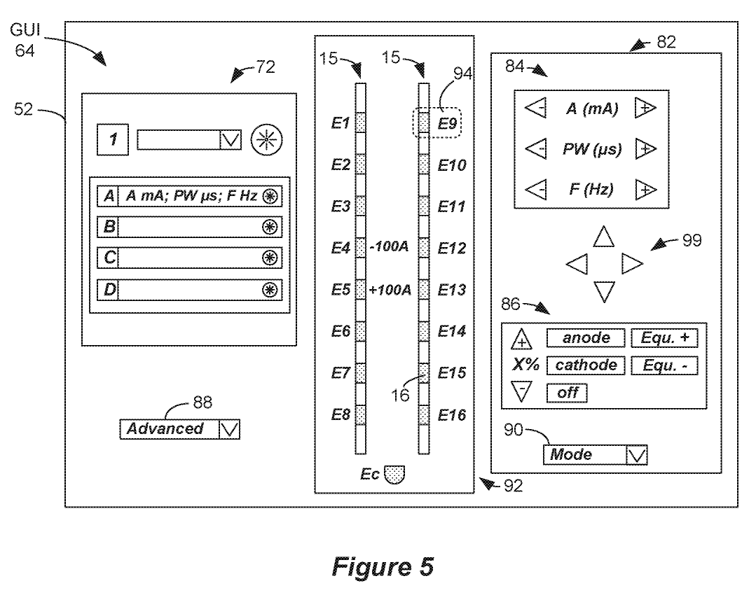

A portion of an example GUI 64 is shown in one example in FIG. 5. One skilled in the art will understand that the particulars of the GUI 64 will depend on where clinician programmer software 66 is in its execution, which will depend on the GUI selections the clinician has made. Some embodiments of the GUI 64 may include a representation of the patient’s vertebrae or some other indication of the vertebral level (e.g., T7, T8, T9, etc.) of the electrode leads/electrodes, but that is not shown in the illustration. FIG. 5 shows the GUI 64 at a point allowing for the setting of stimulation parameters for the patient and for their storage as a stimulation program. To the left a program interface 72 is shown, which as explained further in the ‘038 Publication allows for naming, loading and saving of stimulation programs for the patient. Shown to the right is a stimulation parameters interface 82, in which specific stimulation parameters (A, D, F, E, P) can be defined for a stimulation program. Values for stimulation parameters relating to the shape of the waveform (A; in this example, current), pulse width (PW), and frequency (F) are shown in a waveform parameter interface 84, including buttons the clinician can use to increase or decrease these values.

Stimulation parameters relating to the electrodes 16 (the electrodes E activated and their polarities P), are made adjustable in an electrode parameter interface 86. Electrode stimulation parameters are also visible and can be manipulated in a leads interface 92 that displays the leads 15 (or 15’) in generally their proper position with respect to each other, for example, on the left and right sides of the spinal column. A cursor 94 (or other selection means such as a mouse pointer) can be used to select a particular electrode of the electrode array in the leads interface 92. Buttons in the electrode parameter interface 86 allow the selected electrode (including the case electrode, Ec) to be designated as an anode, a cathode, or off. The electrode parameter interface 86 further allows the relative strength of anodic or cathodic current of the selected electrode, which is specified in terms of a percentage, X, in this example. This is particularly useful if more than one electrode is to act as an anode or cathode at a given time, as explained in the ‘038 Publication. In accordance with the example waveforms shown in FIG. 2, as shown in the leads interface 92, electrode E5 has been selected as the only anode to source current, and this electrode receives X = 100% of the specified anodic current, +A. Likewise, electrode E4 has been selected as the only cathode to sink current, and this electrode receives X = 100% of that cathodic current, -A.

The GUI 64 as shown specifies only a pulse width PW of the first pulse phase 30a. The clinician programmer software 66 that runs and receives input from the GUI 64 will nonetheless ensure that the IPG 10 and ETS 40 are programmed to render the stimulation program as biphasic pulses if biphasic pulses are to be used. For example, the clinician programming software 66 can automatically determine durations and amplitudes for both of the pulse phases 30a and 30b (e.g., each having a duration of PW, and with opposite polarities +A and -A). An advanced menu 88 can also be used (among other things) to define the relative durations and amplitudes of the pulse phases 30a and 30b, and to allow for other more advanced modifications, such as setting of a duty cycle (on/off time) for the stimulation pulses, and a ramp-up time over which stimulation reaches its programmed amplitude (A), etc. A mode menu 90 allows the clinician to choose different modes for determining stimulation parameters. For example, as described in the ‘038 Publication, mode menu 90 can be used to enable electronic trolling, which comprises an automated programming mode that performs current steering along the electrode array by moving the cathode in a bipolar fashion.

Once an SCS patient has had one or more electrode leads implanted in their spinal column, they will typically present to a clinician who will use a programmer, such as clinician programmer 50 equipped with a user interface such as the GUI 64 to determine one or more stimulation programs that are expected to be effective for treating the patient’s indication(s). Each of the stimulation programs comprises a number of parameters, including which electrodes in the electrode array will be activated for providing the stimulation current at a given time, the amplitude of the current to be delivered, an electrode or pole configuration that specifies how the total current amplitude is fractionated among the active electrodes, the frequency of the stimulation, the pulse width of the pulses of the stimulation waveform, whether active or passive recharge will be employed, whether the stimulation waveform will be a tonic waveform, a burst, etc., as well as other parameters relevant to specific stimulation modalities. The process of determining one or more appropriate stimulation programs for a patient is referred to herein as a “fitting session.”

Selecting particular electrodes of the electrode array to deliver the stimulation current effectively determines the location on the patient’s spinal cord stimulation will be delivered. The clinician seeks to provide stimulation at a spinal cord location that alleviates the patient’s pain. In FIG. 5, the electrode E4 is selected to deliver 100% of the cathodic current and E5 is selected to deliver 100% of the anodic current, as mentioned above. During a typical fitting session, the clinician will investigate different electrode combinations for delivering stimulation. This process is referred to herein as a “sweet spot search” because the clinician tries to determine the “sweet spot” where stimulation is most effective.

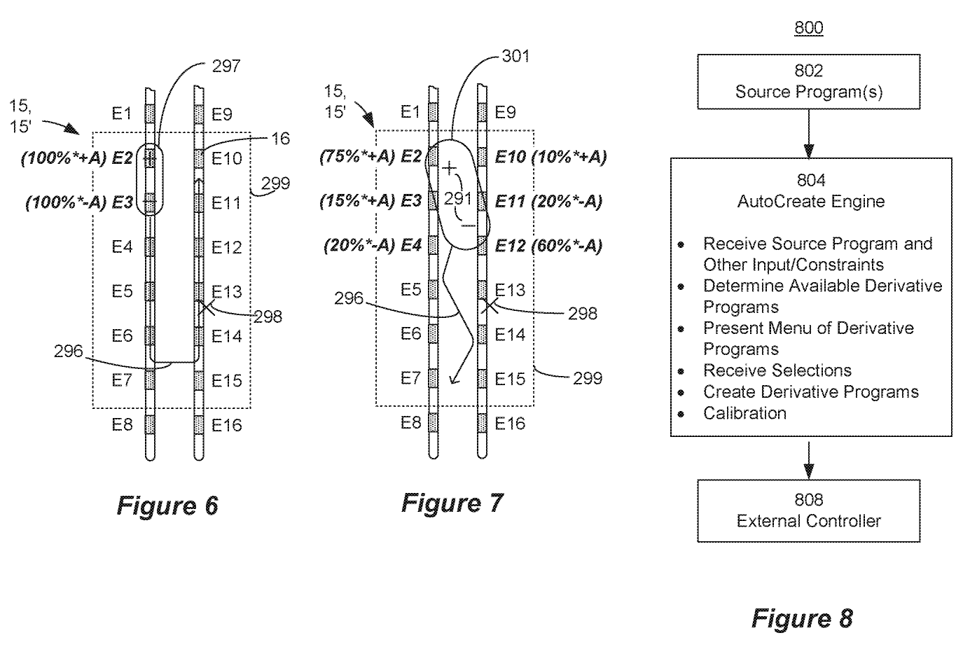

FIG. 6 briefly illustrates a technique for a sweet spot search, i.e., how electrodes can be selected that are proximate to a site of pain in a patient. In the example shown, it is assumed that a site 298 where stimulation will be effective at treating the patient’s pain (i.e., a “sweet spot” for stimulation and the location the clinician is attempting to find) is likely within a tissue region 299. Such region 299 may be deduced by a clinician based on the patient symptoms, e.g., by understanding which electrodes are proximate to certain vertebrae (not shown), such as within the T9–T10 interspace. In the example shown, region 299 is bounded by electrodes E2, E7, E15, and E10, meaning that electrodes outside of this region (e.g., E1, E8, E9, E16) are unlikely to have an effect on the patient’s symptoms. Therefore, these electrodes may not be selected during the sweet spot search depicted in FIG. 6, as explained further below.

In FIG. 6, a bipole 297 is selected, in which one electrode (e.g., E2) is selected as an anode that will source a positive current (+A) to the patient’s tissue, while another electrode (e.g., E3) is selected as a cathode that will sink a negative current (-A) from the tissue. When stimulation is applied using the bipole 297 the patient can tell the clinician if stimulation evokes any sensation, and if so, if the location of that sensation coincides with the location of the patient’s pain. After the bipole 297 is tested at this first location, a different combination of electrodes is chosen (e.g., anode electrode E3, cathode electrode E4), which moves the location of the bipole 297 in the patient’s tissue. In the example shown, the bipole 297 is moved down one electrode lead, and up the other, as shown by path 296 in the hope of finding a combination of electrodes that covers the sweet spot 298.

Notice that the example illustrated in FIG. 6 uses only a single anode electrode and a single cathode electrode. FIG. 7 shows another bipole 301 that may be used, and in particular shows how the virtual bipoles may be formed using virtual poles by activating three or more of the electrodes. Virtual poles are discussed further in USP 10,881,859, which is incorporated herein by reference in its entirety, and thus virtual poles are only briefly explained here. Forming virtual poles is assisted if the stimulation circuitry 28 or 44 used in the IPG or ETS is capable of independently setting the current at any of the electrodes—what is sometimes known as a Multiple Independent Current Control (MICC).

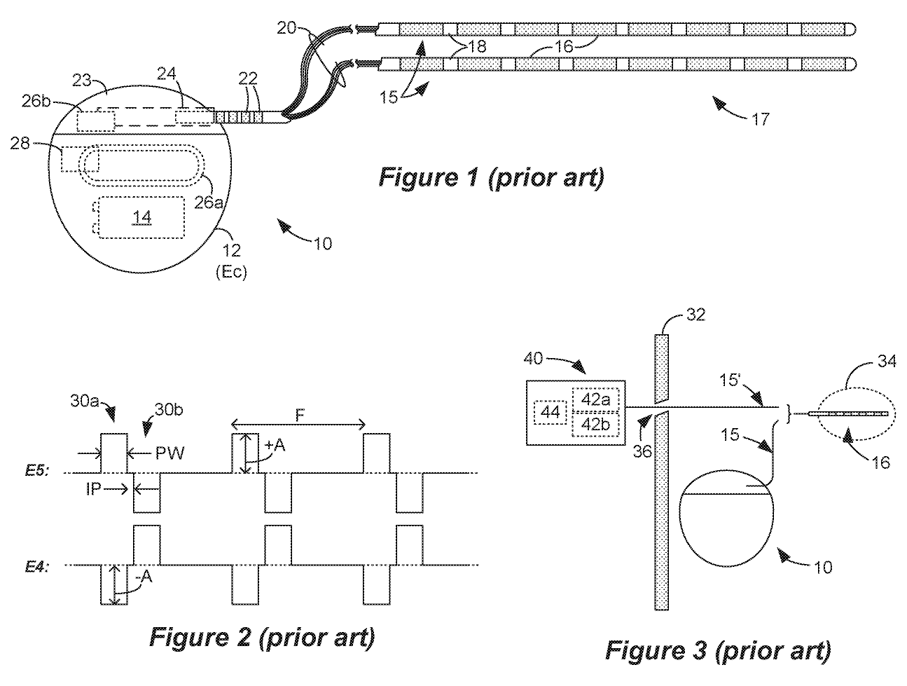

When a virtual bipole is used, the GUI 64 (FIG. 5) of the clinician programmer 50 (FIG. 4) can be used to define an anode pole (+) and a cathode pole (-) at positions 291 (FIG. 7) that may not necessarily correspond to the position of the physical electrodes 16. The control circuitry 70 in the clinician programmer 50 can compute from these positions 291 and from other tissue modeling information which physical electrodes 16 (FIG. 1) will need to be selected and with what amplitudes to form the virtual anode and virtual cathode at the designated positions 291. As described earlier, amplitudes at selected electrodes may be expressed as a percentage X% of the total current amplitude A specified at the GUI 64 of the clinician programmer 50.

For example, in FIG. 7, the virtual anode pole is located at a position 291 between electrodes E2, E3 and E10. The clinician programmer 50 may then calculate based on this position that each of these electrodes (during first pulse phase 30a, FIG. 2) will receive an appropriate share (X%) of the total anodic current +A to locate the virtual anode at this position. Since the virtual anode’s position is closest to electrode E2, this electrode E2 may receive the largest share of the specified anodic current +A (e.g., 75%*+A). Electrodes E3 and E10 which are proximate to the virtual anode pole’s position but farther away receive lesser shares of the anodic current (e.g., 15%*+A and 10%*+A respectively). Likewise, it can be seen that from the designated position 291 of the virtual cathode pole, which is proximate to electrodes E4, E11, and E12, that these electrodes will receive an appropriate share of the specified cathodic current –A (e.g., 20%*-A, 20%*-A, and 60%*-A respectively, again during the first pulse phase 30a). These polarities would then be flipped during the second phases 30b of the pulses. In any event, the use of virtual poles in the formation of bipole 301b allows the field in the tissue to be shaped, and many different combinations of electrodes can be tried during the sweet spot search. In this regard, it is not strictly necessary that the (virtual) bipole be moved along an orderly path 296 with respect to the electrodes, and the path may be randomized, perhaps as guided by feedback from the patient. It is worth noting that the bipoles formed during the sweet spot search (whether simple two-electrode bipoles or virtual bipoles) provide a central points of stimulation (CPS), which is the location where stimulation is most concentrated for a given pole configuration. Thus, another way of thinking about the sweet-spot search is that it is a process of moving the CPS to a location where stimulation is most effective.

Once the appropriate location for stimulation is identified, other stimulation parameters may be determined. These stimulation parameters may relate to properties of the stimulation waveform, such as the amplitude, frequency, pulse width, (as shown in FIG. 2) etc. Of particular interest here is the stimulation amplitude. Traditional SCS typically involves providing a stimulation amplitude that results in stimulation that the patient can perceive at the body location innervated by nerves originating at the spinal cord location being stimulated. Paresthesia is a sensation such as tingling, prickling, heat, cold, etc. that can accompany SCS therapy. Generally, the effects of paresthesia are mild, or at least are not overly concerning to a patient. Moreover, paresthesia is generally a reasonable tradeoff for a patient whose chronic pain has now been brought under control by SCS therapy. Some patients even find paresthesia comfortable and soothing. Such perceptible stimulation is referred to herein as supra-perception (supra-P) stimulation and the minimum stimulation amplitude that elicits such sensations is referred to as the perception threshold (Pth). Generally, the effects of paresthesia are mild, or at least are not overly concerning to a patient. Moreover, paresthesia is generally a reasonable tradeoff for a patient whose chronic pain has now been brought under control by SCS therapy. Some patients even find paresthesia comfortable and soothing. An aspect of the fitting procedure may involve determining Pth for a given set of stimulation parameters. At least for some patients, SCS therapy would ideally provide complete pain relief without paresthesia—what is often referred to as “sub-perception” or sub-threshold (sub-P) therapy that a patient cannot feel (i.e., stimulation with an amplitude that is below Pth).

As explained in U.S. Patent Application Publication 2025/0256112, the entire contents of which are incorporated herein by reference, even if it is intended to ultimately treat a patient with sub-P therapy, it may still be beneficial to perform the sweet-spot search using supra-P stimulation. Using supra-P stimulation during the sweet-spot search allows the patient to provide feedback regarding the location on their body where they perceive the paresthesia evoked by the stimulation, thereby aiding the clinician to find an appropriate stimulation location. Once the sweet-spot is determined using supra-P stimulation the amplitude of the stimulation can be reduced to below the patient’s perception threshold to provide sub-P chronic therapy, if so desired.

The sweet-spot search methodology described above generally arrives at a stimulation program that is expected to treat the patient’s symptoms. Once that program is derived (typically using the clinician programmer 50 (FIG. 4)), the stimulation program may be transferred to the patient’s external controller 45 (FIG. 4), which the patient may use to instantiate the program, and possibly make minor adjustments to aspects of the program. For example, the patient may adjust the stimulation amplitude, depending on how the patient is feeling at a particular time. But it may be desirable to provide the patient with more than one stimulation program. In some cases, it is desirable to configure the patient’s external controller with a menu of stimulation programs from which the patient may select a suitable program for their given circumstances at a particular time. For example, the patient may prefer one program when they are awake and a different program when they are asleep or resting, and yet another program when they are exercising. A patient may prefer one program when their symptoms are especially bothersome and another program when their symptoms are less severe. The patient may prefer the option of choosing a sub-P or a supra-P program. It may also be desirable to configure the patient’s external controller with one or more backup programs that may be instantiated in case the efficacy of the patient’s primary programs declines for some reason.

A person of skill in the art will recognize that optimizing a plurality of stimulation programs may be burdensome to a clinician. The process of deriving each of the programs requires a significant amount of time and may involve many user interactions. The complexities of setting up multiple programs contribute to mistakes within the clinical setting that may result in sub-optimal results for the patient. Aspects of this disclosure provide methods and systems for facilitating the programming of a plurality stimulation programs. According to some embodiments, the user may focus their time and energy on creating one (or a limited number of) source stimulation program and the methods and systems described herein may use the source stimulation program(s) to automatically generate a plurality of derivative stimulation programs.

FIG. 8 illustrates a high-level workflow 800 according to aspects of this disclosure. Various aspects of the workflow will be discussed in more detail below. Briefly, in the illustrated workflow, the user derives one or more source stimulation programs 802, for example, using procedures as described above for determining an appropriate electrode configuration (i.e., stimulation location, CPS) and stimulation parameters such as amplitude, frequency, pulse width, etc. In some embodiments, the source stimulation program may be selected from the clinician programmer 50, the patient’s external controller 45, or the IPG/ETS 10/40. At step 804, parameters of the source stimulation program(s) are provided to a creation engine (referred to herein as an “AutoCreate Engine”). The AutoCreate engine is configured to use parameters of the source stimulation program to generate a plurality of derivative stimulation programs. The AutoCreate engine may also receive other inputs that may constrain or otherwise impact which derivative stimulation programs may be created and/or aspects of such derivative stimulation programs.

The AutoCreate engine is configured to determine, based on the source stimulation program and the other inputs and/or constraints, what derivative stimulation programs may be created. As mentioned above, the available derivative stimulation programs may be constrained based on information provided to the AutoCreate engine, in addition to the source stimulation program. For example, some derivative stimulation programs may not be appropriate in certain situations. For example, if the patient’s IPG is equipped with a primary cell battery instead of a rechargeable battery, then stimulation programs requiring a high energy usage may be less desirable. Also, certain derivative stimulation programs may be contraindicated for certain patient symptoms or indications. In some embodiments, the AutoCreate engine may suggest derivative programs that are expected to be appropriate, given the source program and other inputs/constraints and may restrict other derivative programs (or specific parameters of such derivative programs). In some embodiments, the user may be given the ability to override such restrictions. In some embodiments, the AutoCreate engine may select the available or appropriate derivative stimulation programs from a library of derivative stimulation programs, which may be stored within the external computing device (such as the clinician programmer).

Based on the AutoCreate engine’s determination of the available derivative stimulation programs, the AutoCreate engine may provide a menu of available derivative stimulation programs, for example, by presenting a list of the available derivative stimulation programs on a GUI, such as GUI 64 of the clinician programmer 50. The AutoCreate engine may receive selections from the user of which derivative stimulation programs to create and then take steps to create the specified derivative programs. In some embodiments, this may involve executing scripts and/or other coded algorithms to create each of the derivative stimulation programs. Once the selected programs have been created the AutoCreate engine may instantiate a calibration interface whereby the user may calibrate derivative stimulation programs that require calibration. Calibration may include the setting or adjustment of one or more parameters of the Program, such as the amplitude or dose of stimulation, or the location (fractionalization, field) of stimulation. Different parameters may need to be calibrated depending on the source and derivative program, and not all parameters may need to be calibrated for any given derivative program. Once the derivative stimulation programs are created and calibrated (if needed), they may be transmitted to the patient’s external controller or IPG/ETS (step 808). In other words, the patient’s externa controller, and/or IPG/ETS may be programmed so that the source and derivate programs may be selected and executed to provide the appropriate stimulation to the patient. In some embodiments, this involves saving the derivative stimulation programs in designated memory locations, or “slots,” in the external computing device (e.g., clinician’s programmer 50) and then transferring the programs to corresponding slots of the patient’s external controller 45 or the IPG/ETS so that the stimulation programs may be used to provide stimulation to the patient.

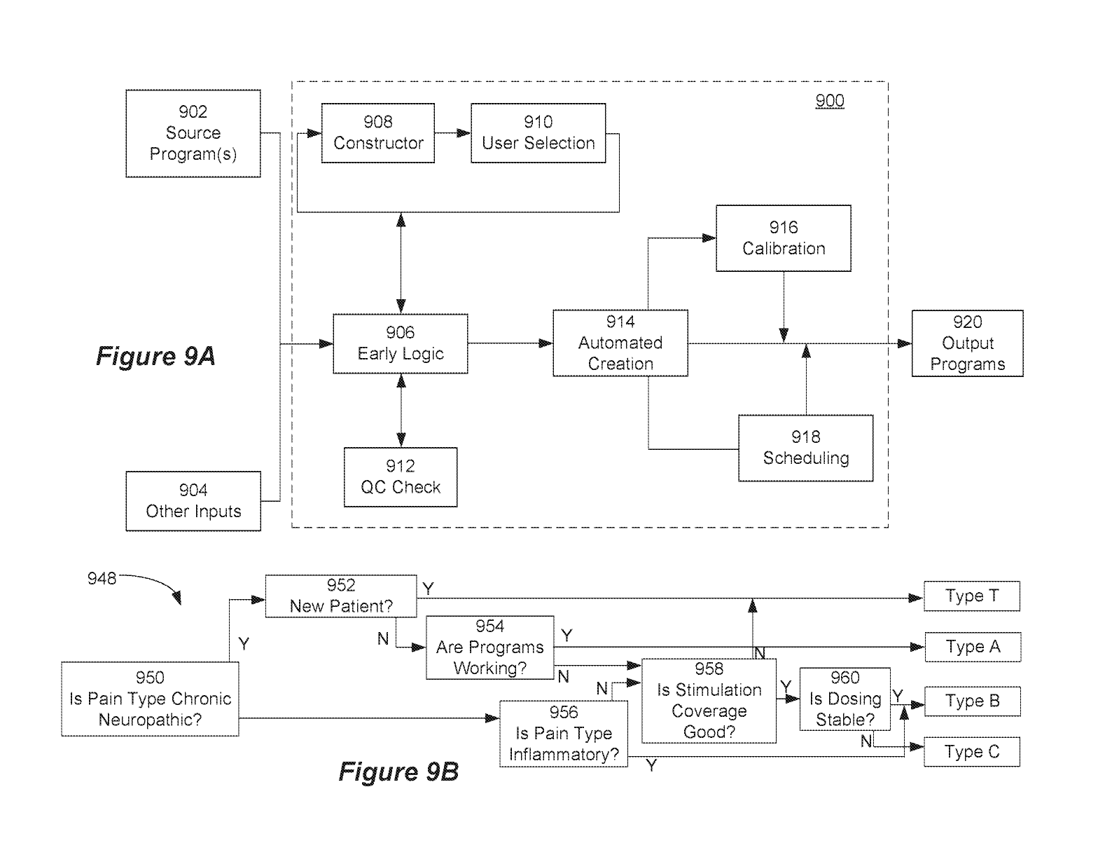

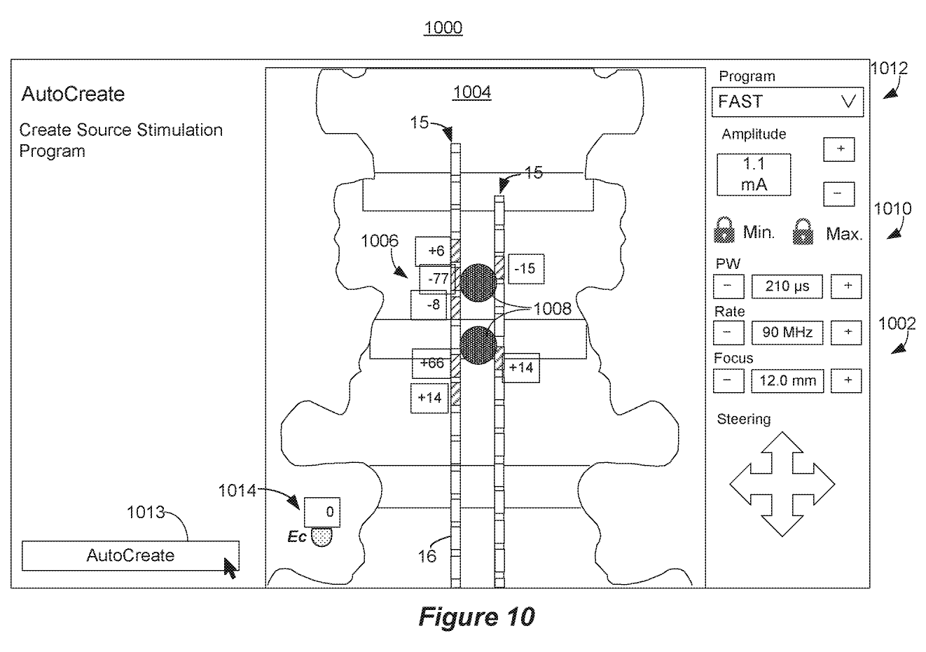

FIG. 9A illustrates a more detailed overview of aspects of implementing the AutoCreate engine 900 during programming a plurality of stimulation programs, as described herein. Step 902 involves providing one or more source stimulation programs. In the illustrated example, assume that a single source program is provided. FIG. 10 illustrates an example of a GUI 1000 that may be used to derive the one or more source stimulation programs. The GUI 1000 is similar to the GUIs 64 (FIG. 5) discussed above and includes GUI elements 1002 for controlling the various parameters of the stimulation, steering the stimulation, etc. The illustrated GUI 1000 also features a graphical representation 1004 of the patient’s spinal anatomy and the location of the electrode lead(s) 15 and electrode contacts 16 with respect to the anatomy. The GUI may include indicators 1006 indicating the electrode configuration, i.e., how stimulation current is fractionated among the active electrodes. The case icon 1014 shows the current being provided at the case electrode (Ec), which is 0 V in the illustrated example. The GUI may also include graphical elements indicating a pole configuration 1008 of the stimulation pole(s) resulting from the electrode fractionalization. The pole configuration is also indicative of the CPS.

In the illustrated example, assume that the clinician has determined the stimulation program illustrated in FIG. 10 by following a process described above (e.g., with reference to FIGS. 6 and 7), and/or a process described in the incorporated ‘944 Application. In some examples, the clinician may use supra-perception stimulation to determine a pole configuration that provides stimulation that readily corresponds to the location on the patient’s body that covers their pain. Once that location is determined, the clinician may reduce the stimulation amplitude to a level where the patient can no longer feel paresthesia (i.e., below the patient’s perception threshold). The clinician may lock the maximum amplitude of the stimulation at a level corresponding to the perception threshold, for example, using the Min. and Max. Lock icons 1010. The illustrated stimulation program corresponds to a symmetric biphasic waveform providing actively driven charge recovery. Sub-P programs of this type are referred to in the incorporated ‘944 Application as Fast Acting Sub-Threshold (“FAST”) (see program selection menu 1012 of instant FIG. 10) and further details for configuring such programs are described therein. Once the source program (the FAST program in the illustrated example) is configured, the user may select the AutoCreate icon 1013 to launch the algorithm(s) for deriving the derivative stimulation programs.

Referring again to FIG. 9A, step 904 may also involve providing other inputs for the AutoCreate functionality. As mentioned above, which may be used to determine which derivative stimulation programs may be created. These other inputs may also impact aspects of the workflow. In other words, some steps of the workflow may or may not be performed based on these other inputs.

The other inputs 904 may relate to specific patient details such as the body location(s) of the patient’s pain, the etiology of the pain (for example, failed back surgery syndrome (FBSS), diabetic peripheral neuropathy (DPN), etc.), the type of pain (for example, nociceptive, neuropathic, inflammatory, mechanical, etc.), and/or other patient details. The other inputs 904 may include treatment and/or targeting information that is known or previously determined about the patient, such as the location of the patient’s physiological midline with respect to their anatomical midline, stimulation amplitude thresholds, tolerances, and/or rankings, etc. Targeting information may include information from previous targeting sessions, such as pain/paresthesia overlap at the central point of stimulation and/or comparison of historical values with the present value. The other inputs 904 may include information about preferences and/or options relating to the programming session or other patient and/or clinician preferences that may impact the types of derivative programs that will be created and/or may impact the workflow of the programming session. Such other inputs may include prioritizations of the goals of the programming session. For example, the patient and/or clinician may prioritize minimizing the time required for the programming session, in which case the workflow may deprioritize the creation of derivative stimulation programs that require extensive calibration. The other inputs may reflect the purpose of the programming session, i.e., whether the patient is a new patient or is a returning patient that wants to explore new therapy options, whether the patient is a returning patient whose therapy has declined, etc.

According to some embodiments, the GUI may provide a check list, menu, survey, questionnaire, or other means to assist the user in providing the other inputs. In some examples, the GUI may instantiate a Wizard, a chat tool such as a chatbot, speech-to-text, or natural language processing tool to receive the other inputs. In some embodiments, the system may be configured to import and parse medical records to create some or all of the other inputs. For example, data from a resource such as Medidata may be downloaded and parsed for diagnosis, phenotype terms, etc., for example, using a large language model (LLM). The other inputs may be derived from programming records and/or ratings/outcome scoring of programs that have been previously tried with the patient.

According to some embodiments, parsed inputs may be classified or clustered to provide insight into the patient’s disease state and/or the purpose of the programming session. The AutoCreate functionality may use this information to tailor the available derivative stimulation programs and/or the programming workflow. FIG. 9B illustrates an example of a classification algorithm 948 for using various inputs to classify a patient into various “types” which may tailor aspects of the programming session workflow based on the expected outcome of the programming session. The illustrated algorithm 948 is not intended to be exhaustive but is instead intended to illustrate how aspects of the other inputs may be used to tailor a programming session. Step 950 involves determining if the patient’s pain is neuropathic. This may be based on electronic medical records (EMR), for example, by identifying words such as neuropathic, radiating, etc. A programming workflow for a new patient (Step 952) experiencing neuropathic pain may be classified as “Type T" meaning that it will focus on targeting (or re-targeting, in the case of a returning patient). Likewise, a returning patient (Step 954) whose present stimulation programs are working may be classified a “Type A” meaning that the programming workflow may be adapted for diagnostic and/or maintenance purposes. A patient not classified as experiencing chronic neuropathic pain may be classified based on whether their pain is inflammatory (Step 956). Again, this may be determined based on parsing EMR data, for example, looking for inflammatory biomarkers and the like, or key words associated with inflammatory pain, such as “all over,” “joints,” etc. A patient with indicators of inflammatory pain may be classified as “Type B” and the programming session may deemphasize stimulation programs that are typically less effective for inflammatory pain (such as FAST) and focus more strongly on programs that target dorsal horn fibers (described below). A returning patient whose present stimulation programs are not working may be tested to see if the coverage of their present stimulation programs is effective (Step 958). For example, is there effective paresthesia/pain overlap? If the answer is no, then that patient may be classified as “Type T" and the programming session may focus on targeting. Historical and/or current values pain-paresthesia overlap and central point of stimulation may be considered during this evaluation. If the answer is yes, the patient may be tested to determine if the dosing of the patient’s stimulation is stable (Step 960). If the dosing is stable, but the patient is still not satisfactory, then the patient may be classified as “Type B” and the programming session may be tailored to focus on alternative therapies, such as dorsal horn stimulation. If the dosing is stable, then the patient may be classified as “Type C” meaning that the programming session may focus on control of habituation.

In some embodiments the algorithm 948 for “typing” patients may involve scoring the various criteria and weighing the scores to derive a “type” for the patient. For example, the patient’s pain type, the efficacy of their present therapy, the extent to which their present stimulation covers their pain (e.g., pain/paresthesia overlap), and their present dosing may each be scored and the respective criteria may be weighted according to the score to classify the patient.

In some embodiments, the other inputs may include information about the patient’s implanted medical device, for example, the number and type(s) of electrode leads implanted in the patient, the number and/or spacings of the electrode contacts on the lead, the type of battery used by the patient’s IPG (i.e., whether it is a primary cell (PC) or rechargeable battery), etc. In some embodiments, aspects of the other inputs may be derived or extracted from the patient’s stimulator and/or their external controller.

In some embodiments, one or more flags or metadata may be associated with the other inputs, or the other inputs may be categorized or sorted according to specified criteria that may be used to match the other inputs with potential derivative stimulation programs that may, or may not, be compatible with a particular instance of other data. In other words, the appropriate derivative stimulation programs may be selected, at least in part, based on the other inputs.

The other inputs 904 along with information from the source stimulation program(s) 902 are provided to a block 906, referred to herein as “early logic.” In some embodiments, the early logic block 906 comprises one or more algorithms configured to gate the programming users access to the autocreate feature, for example for the purpose of ensuring that the user selected program, current program, or otherwise the program designated for use as the source program meets the necessary requirements to be fit for use. In some embodiments, the early logic block 906 comprises one or more algorithms configured to use the source stimulation program 902 and the other inputs 904 to determine which derivative output programs are available. In some embodiments, the AutoCreate engine may comprise a database of potential derivative stimulation programs. Each of the derivative stimulation programs in the database may be associated with flags or metadata that may be compared or parsed against flags or metadata associated with the various “other inputs” provided in block 904 to determine if the potential derivative stimulation program is compatible with each respective other input. In some embodiments the early logic block may work with a constructor block 908 to parse data indicative of the source program and the other inputs with respect to the universe of potential derivative stimulation programs in the database to construct a list of available derivative stimulation programs.

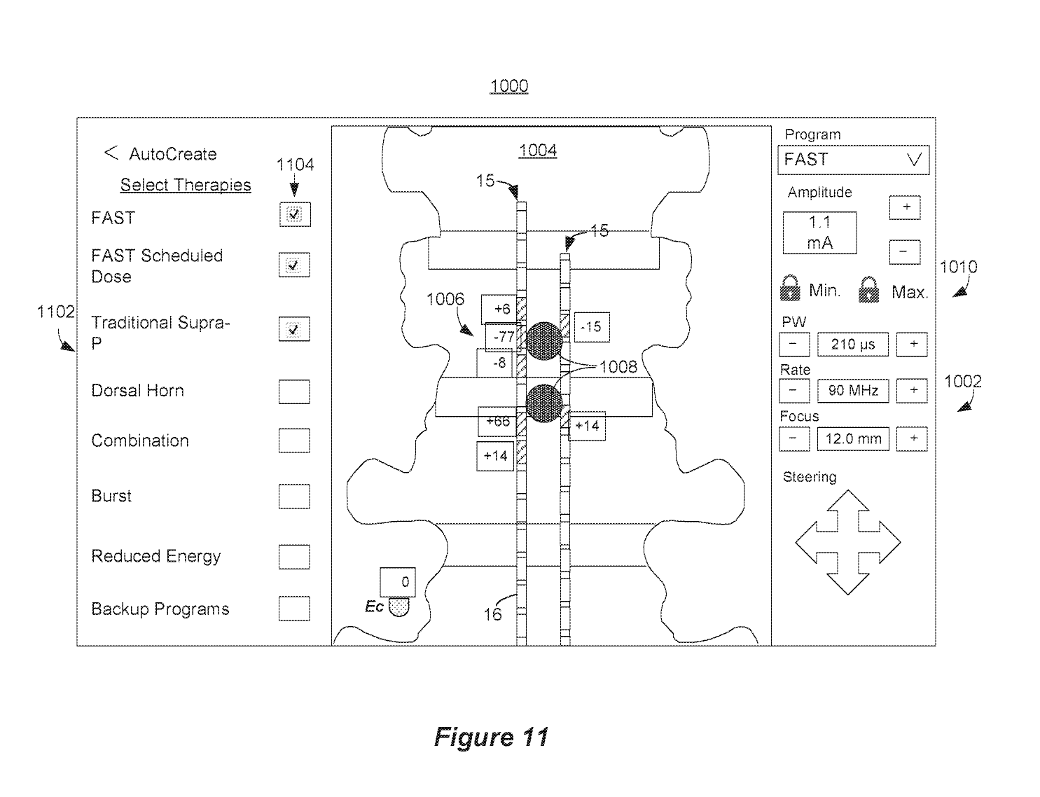

Referring to FIG. 11, the constructor block may instruct the GUI 1000 to display a selectable list (i.e., a menu) of available derivative stimulation programs 1102 for different selectable derivative stimulation programs, as shown in FIG. 11. The user may select the derivative stimulation programs that they wish to create from the list/menu by selecting a box 1104 (or other GUI element). The illustrated embodiment lists six selectable derivative stimulation programs, as well as a reduced energy option and an option to create backup programs.

Here we briefly describe the stimulation programs that appear in the menu 1102, simply for the purposes of illustration. However, it should be appreciated that the disclosed methods and systems are not limited to these illustrated stimulation programs. Generally, any type of stimulation program may be created using the AutoCreate functionality; the AutoCreate functionality must simply be configured with scripts/algorithms for constructing a given stimulation program based on steps that are implemented to modify the source stimulation program, as described in more detail below.

The FAST program appearing in the menu 1102 is simply the source stimulation program described above and described in more detail in the incorporated ‘944 Application. The FAST stimulation program is a sub-P program configured to target neural elements in the dorsal column, and an active recharge (actively driven charge recovery) bipole 1008, as shown in FIG. 11. Dosing of the stimulation (i.e., total charge imparted to the patient’s tissue) may be controlled by adjusting the pulse width and frequency (rate) of the stimulation waveform, as well as the amplitude (typically, as a fraction of the patient’s perception threshold (PT)). Multiple FAST programs may be constructed with different dosing levels, such as a FAST90 program (PW = 210µs, Freq. = 90Hz, Amp. = 30% of PT) and a FAST30 program (PW = 340 µs, Freq. = 30 Hz, Amp. = 50% of PT). These various FAST programs may be presented as options in the menu of derivative programs 1102, though they are not shown in FIG. 11, for brevity.

The illustrated FAST Scheduled Dose program may configure the schedule function of the patient’s external controller to cycle between two or more stimulation programs according to a defined schedule. This is explained in more detail in the ‘944 Application. Briefly, as an example, the FAST Scheduled Dose feature may be implemented to provide a first FAST program for a first duration, followed by a second FAST program for a second a second duration. For example, a first FAST program with an amplitude of 30 % of the patient’s PT might be provided for four hours, followed by a second FAST program with an amplitude of 70 % of the patient's PT provided for 10 minutes.

The illustrated Traditional Supra-P stimulation program may comprise a monopolar electrode configuration that typically targets neural elements in the dorsal column. The location of the CPS, the amplitude, and the frequency of the program may be determined during a calibration procedure, as described in more detail below. Typically, the amplitude is calibrated so that the patient feels paresthesia evoked by the stimulation. A typical frequency is about 40 Hz and a typical pulse width is about 200 µs, though any values of these parameters may generally be used.

The Dorsal Horn stimulation program is a sub-perception stimulation program that typically seeks to provide electrical stimulation that mostly bypasses dorsal column neural elements and stimulates neural elements of the patient’s dorsal horn. The dorsal horn stimulation may use electrode configurations that generate a wider stimulation field compared to the FAST and traditional supra-P programs. In some embodiments wherein the IPG comprises a rechargeable power supply, the dorsal horn stimulation program may implement a PW of about 200 µs and a frequency of about 200 Hz. For lower energy use applications the dorsal horn stimulation program may use a PW of about 300 µs and a frequency of about 50 Hz.

The Combination stimulation program may be a combination of two or more stimulation programs running simultaneously. For example, the combination program may simultaneously provide stimulation according to a traditional sub-P program and a dorsal horn program, or a FAST-type sub-P program and a dorsal horn program.

The Burst stimulation program may provide bursts of stimulation followed by a quiescent period. For example, a burst of stimulation at a high rate (e.g., 450 pulses per second (pps)) may be provided, followed by a quiescent period.

The AutoCreate engine may also be configured to create permutations of one or more of the selectable stimulation programs. For example, the selectable list of available derivative stimulation programs 1102 shown in FIG. 11 includes Reduced Energy programs and Backup Programs. The reduced energy programs may be versions of any of the available stimulation programs that are configured to use less energy than the “standard” version of the program, for example, by using a lower frequency stimulation than that of the “standard” programs. The backup programs are stimulation programs that may be used in case the efficacy of one or more of the standard stimulation programs decreases. In some embodiments the backup programs may be configured with the same stimulation parameters as the standard programs, but they may comprise electrode configurations that move the CPS rostrally or caudally by some distance.

Referring again to FIG. 9A, the user may select one or more of the available stimulation programs (block 910) from the menu and indications of the selected stimulation programs are provided to the early logic 906. In some embodiments, the workflow may comprise one or more quality control (QC) checks 912, for example, to determine if any of the selected programs conflict with each other, if they conflict with any of the other inputs 904 previously entered, etc. In some embodiments the QC Check and early logic may modify or rework downstream processing.

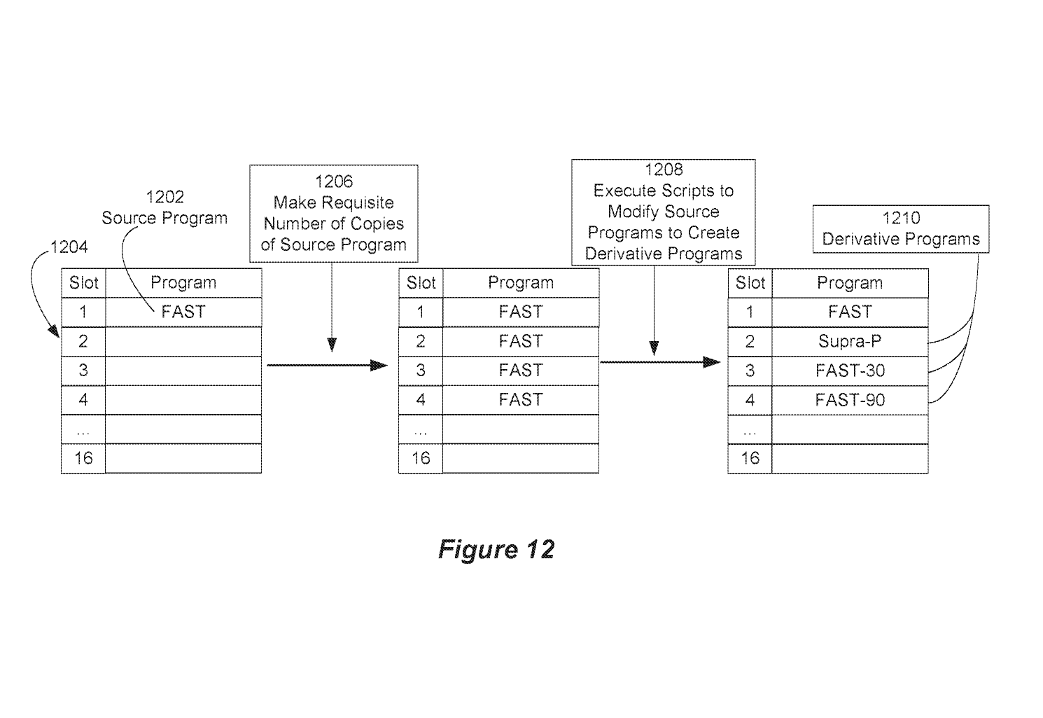

Once the user has selected which available derivative stimulation programs to create the early logic 906 works with a creation algorithm 914 to create the selected derivative stimulation programs. In some embodiments, the early logic 906 and/or the creation algorithm 914 determines a number of derivative stimulation programs that are to be created. The creation algorithm may create a template program for each of the derivative stimulation programs. According to some embodiments, the template program may simply be a copy of the originally created source stimulation program. Referring to FIG. 12, assume that the user has created a FAST stimulation program, as described above, as the source stimulation program 1202. In the illustrated example, the source stimulation program (i.e., FAST) is stored in one of 16 memory slots 1204 (though the number of available memory slots may vary). In the illustrated example, the source stimulation program is stored in slot 1. Assume that the user has selected to create a FAST Scheduled Dose program and a Traditional Supra-P program as derivative programs, as illustrated in FIG. 10. In this case, the early logic 906 and/or the creation algorithm 914 determine that three additional copies of the FAST source stimulation program will be needed to create those derivative stimulation programs. The creation algorithm 914 may execute programs or scripts which cause copies of the source stimulation program to be automatically created and saved into additional memory slots, as shown in step 1206.

Once the requisite number of copies of the source stimulation programs have been created and saved into memory slots, at step 1208 the creation algorithm may execute scripts to modify the copies of the source stimulation programs to create the selected derivative stimulation programs 1210. As mentioned above, the particular scripts may be determined based on the particular source stimulation program being used, the selections the user made from the menu of available derivative stimulation programs 1102 (FIG. 11), as well as other inputs 904 (FIG. 9). For example, the AutoCreate may select different scripts to create the FAST Scheduled Dose derivative stimulation program depending on whether the other inputs indicate that the treatment is for FBSS or DPN. Likewise, different scripts may be used to create reduced energy or standard versions of the derivative stimulation programs. In a similar manner, different scripts may be used depending on the stimulator type, including differentiating between PC and RC IPG or IPG and EPG/ETS, as well as the stimulator edition or generation, or the peripherals that the patient will use, including the RC or RC App. In sum, the other inputs inform which particular script is instantiated to modify the particular source stimulation program to yield the desired derivative stimulation program.

In some embodiments, the scripts for each derivative program essentially automate the steps that a user would make were they to create the derivative stimulation program using the source stimulation program as a starting point. For example, consider the process of reforming the FAST source stimulation program that is saved in the memory slot 2 into the selected Supra-P program. In the illustrated example, the FAST stimulation program is configured with a bipolar electrode configuration such as shown in FIG. 11. The pulse width is 210 µs, the frequency is 90 MHz, and the maximum amplitude is locked at the patient’s perception threshold. The automated creation algorithm 914 (FIG. 9) may be configured to instantiate one or more scripts configured to perform steps including renaming the program in slot 2, setting the maximum amplitude to a higher value (such as the highest safe value that the IPG can provide), reconfiguring the electrode configuration to provide monopolar stimulation, resetting the frequency and pulse width (for example, to about 40 Hz and 200 µs, respectively). In some examples, the script(s) may also configure the derivative stimulation program into a state that is ready for further calibration, as explained further below. For example, the amplitude of the derivative Supra-P stimulation program may be decreased to zero, or near zero, in preparation for further calibration. In prior art embodiments, a user could have made each of these changes manually using the GUI 1000. But the AutoCreate engine and the scripts automatically make these changes at the push of a button.

In the illustrated example, the user has also selected for the AutoCreate engine to create a FAST Scheduled Dose derivative stimulation program. Recall that the FAST Scheduled Dose program provides a first FAST program to be applied for a first duration, followed by a second FAST program applied for a second a second duration. Thus, the automated creation algorithm 914 copied two FAST programs, one each into slots 3 and 4, to form the basis of the FAST Scheduled Dose derivative program. Assume for this example, that the other inputs 904 (FIG. 9) have indicated that the FAST Scheduled Dose program should cycle between FAST-30 and FAST-90 programs. In this case, the creation algorithm may instantiate a first script that reforms the FAST program stored in slot 3 to make a FAST-30 program, for example, by reducing the amplitude to 50 % of the patient’s perception threshold, setting the pulse width to 340 µs, and setting the frequency to 30 Hz. Likewise, the creation algorithm may instantiate a second script that reforms the FAST program stored in slot 4 to make a FAST-90 program, for example, by reducing the amplitude to 30 % of the patient’s perception threshold, setting the pulse width to 210 µs, and setting the frequency to 90 Hz.

Again, it should be noted that this discussion provides examples of how a particular source stimulation program (FAST) can be modified to yield two particular derivative stimulation programs (Supra-P and FAST Scheduled Dose). But generally, the AutoCreate engine may be configured to derive any number of derivative stimulation programs using any source stimulation program as a starting point. The AutoCreate engine simply needs to be configured with appropriate scripts for automating the modification process by automating the particular steps the user would execute manually, as well as the logic to determine whether and how to present those options to the user.

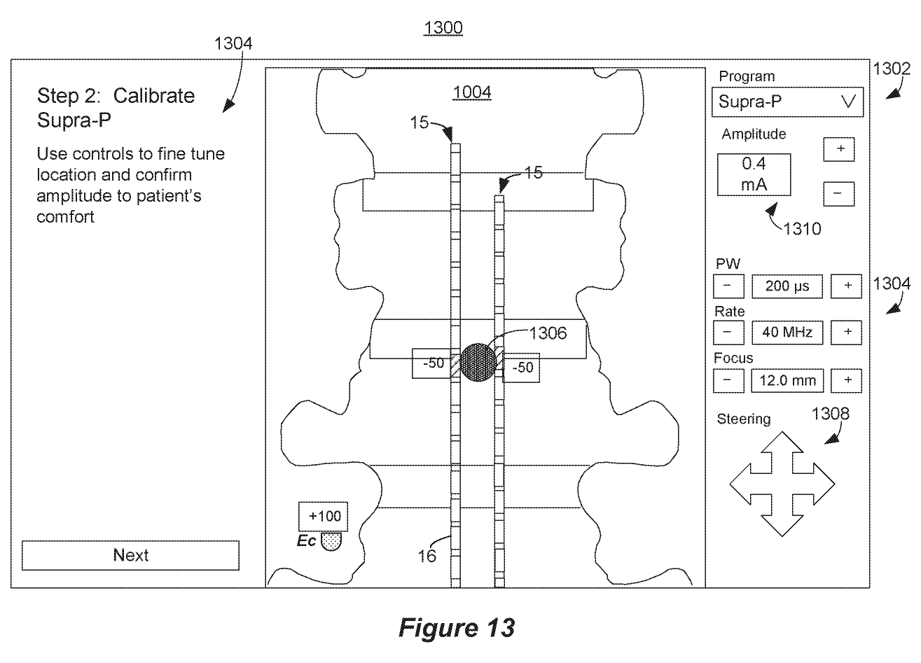

Referring again to FIG. 9A, some of the programs (either the source stimulation program(s) or one or more of the derivative stimulation programs) may require calibration. For example, the Supra-P derivative stimulation program described above is an example of a program that may be calibrated after it is created by the AutoCreate engine. For such programs requiring calibration, a calibration algorithm 916 may instantiate a calibration interface 1300, as shown in FIG. 13. In the illustrated example, the calibration interface may be used to calibrate the Supra-P stimulation program resident in the memory slot 2 (FIG. 12). Notice that in the illustrated interface 1300, the program name 1302 has been assigned as Supra-P and the stimulation parameters 1304 have been configured according to the script described above to be suitable for traditional supra-P stimulation. Also notice that script described above has reconfigured the electrode configuration to provide a single cathodic target pole 1306 with the anodic current being supplied by the case electrode Ec. The user may adjust the pole configuration during the calibration process, for example, using the target pole steering GUI elements 1308. The user may also adjust the amplitude of the stimulation using the amplitude GUI elements 1310. The calibration interface 1300 may provide instructions 1312 to guide the user during the calibration process. Once the calibration process for a program is completed the calibrated settings are saved with the program in its respective memory slot.

In some examples, the user may have selected more than one derivative stimulation program that requires calibration. In such cases embodiments of the AutoCreate engine may create each of those programs and then sequentially guide the user through the calibration procedure for each of the derivative stimulation programs. This streamlined approach may be less confusing, less error-prone, and faster than a scenario in which the user must individually create and then calibrate a program before moving on to a subsequent program.

Referring again to FIG. 9A, some of the programs may require scheduling. For example, the FAST Scheduled Dose derivative stimulation program described above cycles between a FAST-90 program (for example, provided for about 4 hours) and a FAST-30 program (for example, provided for about 10 minutes). In some embodiments, the scheduling (e.g., on/off times for each of the constituent programs) may be selected by the scripts used to create the scheduled dose program. In some embodiments the AutoCreate engine may instantiate a scheduling interface, whereby the user may select or change the scheduling aspects of the scheduled dose programs.

Once the source stimulation program(s) and the derivative stimulation program(s) have been created (and calibrated and/or scheduled, as need be) the stimulation programs may be output to the patient’s device (step 920). Typically, the stimulation programs will be output to the patient’s external controller 45 (FIG. 4) whereby the patient may select programs to run. In some embodiments, the programs contained in the memory slots of the AutoCreate engine/clinician programmer are transmitted to corresponding memory slots of the patient’s external controller.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A system for programming a neurostimulator for providing neurostimulation for a patient, wherein the neurostimulator is configured to connect to one or more electrode leads implantable in the patient, the one or more electrode leads each comprising a plurality of electrodes, the system comprising:
an external programming device comprising control circuitry configured to:
receive a source stimulation program,
receive an indication of one or more derivative stimulation programs which are different from the source stimulation program,
for each of the one or more derivative stimulation programs, automatically cause the external device to make a template stimulation program,
for each template stimulation program, automatically execute steps to modify the template stimulation program to yield one of the indicated derivative stimulation programs, and
cause the external programming device to transmit at least the one or more derivative stimulation programs to an external controller for the neurotransmitter.

2. The system of claim 1, wherein one or more parameters of one or more of the derivative programs is determined based on parameters of the source stimulation program.

3. The system of claim 1, wherein the control circuitry is configured to cause the external programming device to present a graphical user interface (GUI) comprising control elements configured to allow a user to create and modify the source stimulation program;
wherein the GUI comprises a menu of available derivative stimulation programs,
wherein the control circuitry is configured to generate the indication of the one or more derivative stimulation programs in response to user selections from the menu,
wherein the indication of one or more derivative stimulation programs is responsive to user selections from the menu, and
wherein the available derivative stimulation programs are determined, at least in part, in response to one or more inputs.

4. The system of claim 3, wherein the control circuitry is configured to associate metadata with each of the one or more inputs and to use the metadata to select the available derivative stimulation programs.

5. The system of claim 3, wherein the one or more inputs comprises information about the neurostimulator.

6. The system of claim 3, wherein the one or more inputs comprises information about the patient.

7. The system of claim 3, wherein the GUI is configured to guide a user through a process of entering the one or more inputs.

8. The system of claim 7, wherein guiding the user comprises displaying one or more selectable inputs indicative of the one or more inputs or using a chat tool to present questions to the user and to receive answers to the questions.

9. The system of claim 1, wherein the source stimulation program comprises an electrode configuration specifying a fraction of stimulation current provided at each electrode and a total amplitude of the stimulation current.

10. The system of claim 9, wherein the stimulation program specifies a total amplitude corresponding to the patient’s perception threshold.

11. The system of claim 9, wherein the source stimulation program is a sub-perception (sub-P) stimulation program.

12. The system of claim 11, wherein at least one of the one or more derivative stimulation programs is a supra-perception (supra-P) stimulation program.

13. The system of claim 6, wherein modifying the copy of the source stimulation program comprises executing a script that automatically emulates one or more steps that a user would perform using the GUI to modify the copy of the source stimulation program to create the respective derivative stimulation program.

14. The system of claim 13, wherein the one or more steps comprise changing an electrode configuration, wherein the electrode configuration specifies a fraction of stimulation current provided at each electrode.

15. The system of claim 13, wherein the one or more steps comprise moving a center point of stimulation (CPS).

16. The system of claim 13, wherein the one or more steps comprise changing a total amplitude of stimulation current.

17. The system of claim 13, wherein the source stimulation program is a bipolar stimulation program and wherein the one or more steps comprise changing the stimulation to monopolar stimulation.

18. The system of claim 3, wherein the control circuitry is configured to cause the GUI to present a calibration interface whereby the user can calibrate one or more of the derivative stimulation programs.

19. The system of claim 18, wherein the control circuitry is configured to calibrate one or more of an amplitude, stimulation field, pulse width, and/or rate of one or more of the derivate stimulation programs.

20. The system of claim 3, wherein the control circuitry is configured to cause the GUI to present a calibration interface whereby the user can compose a schedule comprising a first duration during which a first of the source or derivative stimulation programs is active to provide stimulation and a second duration during which a second of the source or derivative stimulation programs is active to provide stimulation.
